(19) United States
(12) Patent Application Publication
(10) Pub. No.: US 2006/0211711 A1
(43) Pub. Date: Sep. 21, 2006

(54) 7-(Alkinylamino)-triazolopyrimidines, methods for the production and use thereof to combat harmful fungi and agents containing said compounds

(76) Inventors: Jordi Tormo i Blasco, Laudenbach (DE); Carsten Blettner, Mannheim (DE); Bernd Müller, Frankenthal (DE); Markus Gewehr, Kastellaun (DE); Wassilios Grammenos, Ludwigshafen (DE); Thomas Grote, Wachenheim (DE); Andreas Gypser, Mannheim (DE); Joachim Rheinheimer, Ludwigshafen (DE); Peter Schäfer, Ottersheim (DE); Frank Schieweck, Hessheim (DE); Anja Schwögler, Mannheim (DE); Maria Scherer, Landau (DE); Siegfried Strathmann, Limburgerhof (DE); Ulrich Schöfl, Bruhl (DE); Reinhard Stierl, Freinsheim (DE)

Correspondence Address: BIRCH STEWART KOLASCH & BIRCH, PO BOX 747, FALLS CHURCH, VA, 22040-0747, US

(21) Appl. No.: 10/550,571

(22) PCT Filed: Mar. 30, 2004
(86) PCT No.: PCT/EP04/03346

§ 371 Date: Sep. 23, 2005

(30) Foreign Application Priority Data
Apr. 2, 2003 (DE) 103 14 930.9

(51) Int. Cl. A61K 31/519; C07D 487/04; A01N 43/90

(52) U.S. Cl. 514/259.31; 544/263

(57) ABSTRACT

7-alkinylamino-triazolopyrimidins of formula (I), wherein the substituents have the following meanings: L=halogen, alkyl, halogenalkyl, alkoxy, amino, NHR, NR₂, cyano, S(O)ₙA¹ or C(O)A²; R=alkyl or alkylcarbonyl, A¹=hydrogen, hydroxy, alkyl, alkylamino or aialkylamino; n=0, 1 or 2; A²=alkenyl, alkoxy, halogenalkoxy or one of the groups cited in A¹; m=1, 2, 3, 4 or 5, whereby at least one group L is present in an ortho-position to the bond with the triazolopyrimidine skeleton; X=halogen, cyano, alkyl, halogenalkyl or alkoxy; R¹=hydrogen or alkyl; R²=alkinyl which can be unsubstituted or substituted according to the description. The invention also relates to methods for the production of said compounds, agents containing said compounds and the use thereof to combat harmful phytopathogenic fungi.

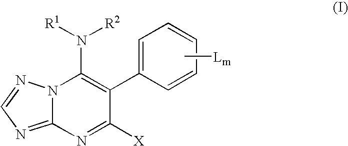

The present invention relates to 7-(alkynylamino)triazolopyrimidines of the formula I 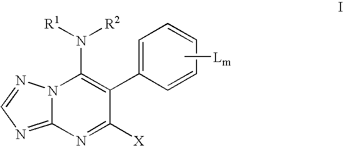
in which the substituents have the following meanings:
L is, independently of one another, halogen, C₁-C₆-alkyl, C₁-C₆-haloalkyl, C₁-C₆-alkoxy, amino, NHR, NR₂, cyano, S(O)ₙA¹ or C(O)A²;
   R is C₁-C₈-alkyl or C₁-C₈-alkylcarbonyl;
   A¹ is hydrogen, hydroxyl, C₁-C₈-alkyl, C₁-C₈-alkylamino or di(C₁-C₈-alkyl)amino;
   n is 0, 1 or 2;
   A² is C₂-C₈-alkenyl, C₁-C₈-alkoxy, C₁-C₆-haloalkoxy or one of the groups mentioned in A¹;
m is 1, 2, 3, 4 or 5, at least one L group being in the ortho position with respect to the bond with the triazolopyrimidine skeleton;
X is halogen, cyano, C₁-C₄-alkyl, C₁-C₄-haloalkyl or C₁-C₄-alkoxy;
R¹ is hydrogen or C₁-C₄-alkyl;
R² is C₃-C₁₀-alkynyl, which can be unsubstituted or partially or completely halogenated or can carry one to three R^{a} groups:
   R^{a} is halogen, cyano, nitro, hydroxyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di(C₁-C₆-alkyl)amino, C₂-C₆-alkenyl, C₂-C₆-alkenyloxy, C₃-C₆-alkynyloxy or C₃-C₆-cycloalkyl,
      these aliphatic or alicyclic groups for their part being able to be partially or completely halogenated or to carry one to three R^{b} groups;
      R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl or dialkylaminothiocarbonyl, the alkyl groups in these radicals comprising 1 to 6 carbon atoms and the abovementioned alkenyl or alkynyl groups in these radicals comprising 2 to 8 carbon atoms.

In addition, the invention relates to processes for the preparation of these compounds, preparations comprising them and their use in the control of harmful phytopathogenic fungi.

6-Phenyl-7-aminotriazolopyrimidines are generally known from EP-A 71 792 and EP-A 550 113. The compounds disclosed in the abovementioned documents are known for the control of harmful fungi.

However, in many cases, their action is unsatisfactory.

It is an object of the present invention to provide compounds which have an improved action and/or a broadened spectrum of activity.

We have found that this object is achieved by the compounds defined at the start. Furthermore, processes for and intermediates in their-preparation, preparations comprising them and methods for the control of harmful fungi with the use of the compounds I have been found.

The compounds of the formula I are distinguished from those from the abovementioned documents by the form of the substitution of the 6-phenyl group, which has to be substituted in the ortho position.

The compounds of the formula I have, in comparison with the known compounds, an increased effectiveness against harmful fungi.

The compounds according to the invention can be obtained in various ways. They are advantageously obtained by reaction of dihalotriazolopyrimidines of the formula II, in which Hal is a halogen atom, such as bromine or, in particular, chlorine, with amines of the formula III under conditions generally known from WO 98/46608.

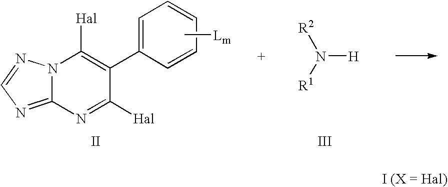

The reaction of II with amines III is advantageously carried out at 0° C. to 70° C., preferably 10° C. to 35° C., preferably in the presence of an inert solvent, such as ethers, e.g. dioxane, diethyl ether or, in particular, tetrahydrofuran, halogenated hydrocarbons, such as dichloromethane, and aromatic hydrocarbons, such as, for example, toluene.

The use of a base, such as tertiary amines, for example triethylamine, or inorganic bases, such as potassium carbonate, is preferred; excess amine of the formula III can also act as base.

Amines of the formula III are known in some cases or can be prepared according to known methods, for example from the corresponding alcohols via the tosylates and phthalimides [cf. J. Am. Chem. Soc., Vol. 117, p. 7025 (1995); WO 93/20804], by reduction of the corresponding nitriles [cf. Heterocycles, Vol. 35, p. 2 (1993); Synthetic Commun., Vol. 25, p. 413 (1995); Tetrahedron Lett., p. 2933 (1995)] or reductive amination of ketones [cf. J. Am. Chem. Soc., Vol. 122, p. 9556 (2000); Org. Lett., p. 731 (2001); J. Med. Chem., p. 1566 (1988)], from the corresponding halides [cf. Synthesis, p. 150 (1995)] and if necessary from subsequent alkylation. The R² group can optionally be formed by a Grignard reaction with corresponding nitriles or carboxylic acid anhydrides [cf. J. Org. Chem., p. 5056 (1992); Tetrahedron Lett., p. 2933 (1995)].

Compounds of the formula I in which X represents cyano or C₁-C₄-alkoxy (formula I.B) can advantageously be prepared from compounds I in which X represents halogen [Hal], preferably chlorine, which correspond to formula I.A.

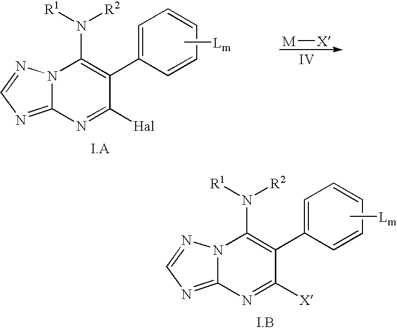

Compounds I.A are reacted with compounds M-X′ (formula IV) to give compounds I.B. Compounds IV represent, depending on the meaning of the X′ group to be introduced, an inorganic cyanide or an alkoxide. The reaction is advantageously carried out in the presence of an inert solvent. The cation M in the formula IV has little meaning; for practical reasons, ammonium, tetraalkylammonium, alkali metal or alkaline earth metal salts are usually preferred.

The reaction temperature is usually from 0 to 120° C., preferably from 10 to 40° C. [cf. J. Heterocycl. Chem., Vol. 12, pp. 861-863 (1975)].

Suitable solvents include ethers, such as dioxane, diethyl ether and, preferably, tetrahydrofuran, halogenated hydrocarbons, such as dichloromethane, and aromatic hydrocarbons, such as toluene.

Compounds I in which X is C₁-C₄-alkyl (formula I.C) can advantageously be prepared by the routes outlined below starting from starting materials of the formula I.A.

Compounds of the formula I.C in which X″ represents C₁-C₄-alkyl can be obtained by coupling 5-halotriazolopyrimidines of the formula I.A with organometallic reagents of the formula V. In one embodiment of this process, the reaction is carried out under transition metal catalysis, such as Ni or Pd catalysis.

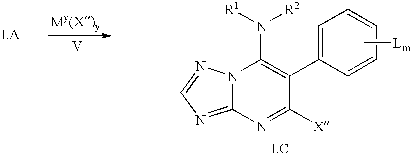

In formula V, X″ is C₁-C₄-alkyl and M is a metal ion with the valency y, such as, for example, B, Zn or Sn. This reaction can, for example, be carried out analogously to the following methods: J. Chem. Soc. Perkin Trans., 1, 1187 (1994), ibid., 1, 2345 (1996); WO 99/41255; Aust. J. Chem., Vol. 43, p. 733 (1990); J. Org. Chem., Vol. 43, p. 358 (1978); J. Chem. Soc. Chem. Commun., p. 866 (1979); Tetrahedron Lett., Vol. 34, p. 8267 (1993); ibid., Vol. 33, p. 413 (1992).

Compounds of the formula I in which X is C₁-C₄-alkyl or C₁-C₄-haloalkyl (formula I.C) can advantageously also be obtained by the following synthetic route:

5-Alkyl-7-hydroxy-6-phenyltriazolopyrimidines VIII are obtained starting from 5-aminotriazole VI and the ketoester VII. In formula VII, R is a C₁-C₄-alkyl group, in particular methyl or ethyl. The 5-methyl-7-hydroxy-6-phenyltriazolopyrimidines are obtained by use of the readily accessible 2-phenylacetoacetic acid esters VIIa with X″═CH₃ [cf. Chem. Pharm. Bull., 9, 801 (1961)]. 5-Aminotriazole VI is commercially available. The starting compounds VII are advantageously prepared under the conditions known from EP-A 1 002 788.

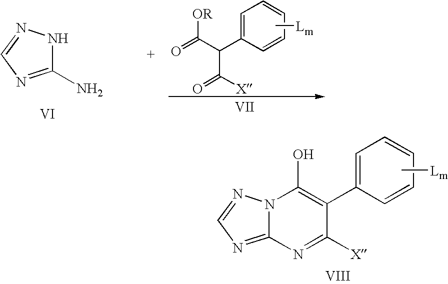

The 5-alkyl-7-hydroxy-6-phenyltriazolopyrimidines VIII thus obtained are reacted with halogenating agents [HAL] to give 7-halotriazolopyrimidines of the formula IX.

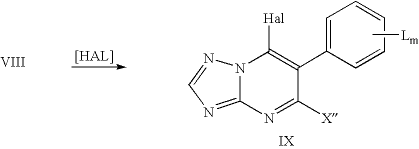

Chlorinating or brominating agents, such as phosphoryl bromide, phosphoryl chloride, thionyl chloride, thionyl bromide or sulfuryl chloride, are preferably used. The reaction can be carried out neat or in the presence of a solvent. Normal reaction temperatures are from 0 to 150° C. or, preferably, from 80 to 125° C.

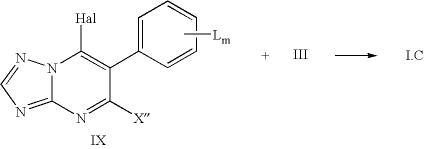

The reaction of 1× with amines III is advantageously carried out at 0° C. to 70° C., preferably 10° C. to 35° C., preferably in the presence of an inert solvent, such as ethers, e.g. dioxane, diethyl ether or, in particular, tetrahydrofuran, halogenated hydrocarbons, such as dichloromethane, and aromatic hydrocarbons, such as, for example, toluene [cf. WO 98/46608].

The use of a base, such as tertiary amines, for example triethylamine, or inorganic bases, such as potassium carbonate, is preferred; excess amine of the formula III can also act as base.

Compounds of the formula I.C can alternatively also be prepared from compounds I.A and malonates of the formula XI. In formula XI, X′″ represents hydrogen, C₁-C₃-alkyl or C₁-C₃-haloalkyl and R represents C₁-C₄-alkyl. They are reacted to give compounds of the formula XII and decarboxylated to give compounds I.C [cf. U.S. Pat. No. 5,994,360].

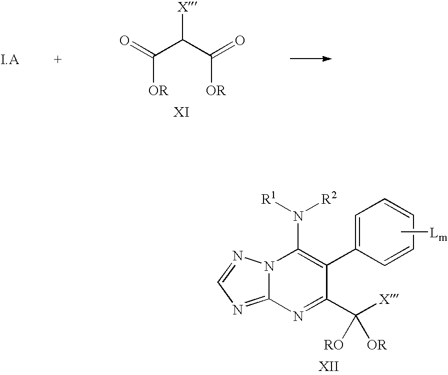

The malonates XI are known in the literature [J. Am. Chem. Soc., Vol. 64, 2714 (1942); J. Org. Chem., Vol. 39, 2172 (1974); Helv. Chim. Acta, Vol. 61, 1565 (1978)] or can be prepared according to the cited literature.

The subsequent saponification of the ester XII is carried out under generally conventional conditions; the basic or the acidic saponification of the compounds XII may be advantageous, depending on the various structural elements. Under the conditions of the ester saponification, the decarboxylation to give I.C may already be completely or partially carried out.

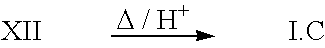

The decarboxylation is usually carried out at temperatures of 20° C. to 180° C., preferably 50° C. to 120° C., in an inert solvent, optionally in the presence of an acid.

Suitable acids are hydrochloric acid, sulfuric acid, phosphoric acid, formic acid, acetic acid or p-toluenesulfonic acid. Suitable solvents are water, aliphatic hydrocarbons, such as pentane, hexane, cyclohexane and petroleum ether, aromatic hydrocarbons, such as toluene or o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitrites, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and dimethyl sulfoxide, dimethylformamide and dimethylacetamide; the reaction is particularly preferably carried out in hydrochloric acid or acetic acid. Mixtures of the abovementioned solvents can also be used.

The reaction mixtures are worked up conventionally, e.g. by mixing with water, separating the phases and possibly chromatographic purification of the crude products. Some of the intermediates and final products are obtained in the form of colorless or slightly brownish viscous oils which, under reduced pressure and at moderately elevated temperature, are freed from or purified of volatile constituents. Provided that the intermediates and final products are obtained as solids, the purification can also take place by recrystallization or trituration.

If individual compounds I are not accessible by the routes described above, they can be prepared by derivatization of other compounds I.

If mixtures of isomers are obtained in the synthesis, a separation is generally not absolutely essential, however, since the individual isomers can sometimes be converted into one another during the workup for the application or in the application (e.g. under the action of light, acid or base). Appropriate conversions can also take place after the application, for example, with the treatment of plants, in the treated plants or in the harmful fungi to be controlled.

Collective terms were used in the definitions of the symbols given in the above formulae, which collective terms are generally representative of the following substituents:

halogen: fluorine, chlorine, bromine and iodine;

alkyl: saturated, straight-chain or branched hydrocarbon radicals with 1 to 4, 6 or 8 carbon atoms, e.g. C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

haloalkyl: straight-chain or branched alkyl groups with 1 to 2 or 4 carbon atoms (as mentioned above), in which the hydrogen atoms in these groups can be partially or completely replaced by halogen atoms as mentioned above: in particular C₁-haloalkyl, such as chloromethyl, bromomethyl, dichloromethyl, trichloromethyl, fluoromethyl, difluoromethyl, trifluoromethyl, chlorofluoromethyl, dichlorofluoromethyl or chlorodifluoromethyl;

alkynyl: straight-chain or branched hydrocarbon groups with 2 to 4, 6, 8 or 10 carbon atoms and one or two triple bonds in any position, e.g. C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

If R² exhibits a chiral center, the (R)- and (S)-isomers and the racemates of the compounds of the formula I come within the scope of the invention.

The embodiments of the intermediates which are especially preferred with regard to the variables correspond to those of the radicals Lₘ, R¹, R² and X of the formula I.

In view of the intended use of the triazolopyrimidines of the formula I, the following meanings of the substituents, in each case alone or in combination, are especially preferred:

Particular preference is given to compounds I in which R¹ represents hydrogen.

Preference is similarly given to compounds I in which R¹ is methyl or ethyl.

Compounds of the formula I in which the R² group exhibits branching in the α-position (formula I.1) are a preferred object of the invention:

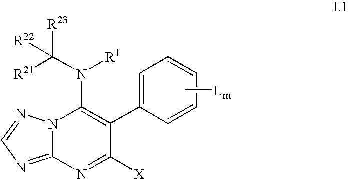

In this connection, R²¹ represents methyl or halomethyl, R²² represents hydrogen, methyl or halomethyl and R²³ represents C₂-C₈-alkynyl which can be unsubstituted or partially or completely halogenated and/or can carry one to three R^{a} groups. The remaining variables are defined as in formula I.

Particular preference is given to compounds I.1 in which R²³ is straight-chain or branched C₂-C₈-alkynyl which can be unsubstituted or partially or completely halogenated and/or can carry one to three C₁-C₃-alkoxy groups. A particularly preferred object are compounds I in which R²³ is straight-chain or branched C₂-C₈-alkynyl which is unsubstituted or partially or completely halogenated.

An additional preferred object of the invention are compounds of the formula I in which R² is one of the following groups: CH₂C≡CH, CH₂C≡CCH₃, CH₂C≡CCH₂Cl, CH₂C≡CCH₂CH₃, CH₂CH₂C≡CH, CH₂CH₂C≡CCH₃, CH₂CH₂C≡CCH₂CH₃, CH₂CH₂CH₂C≡CH, CH₂CH₂CH₂C≡CCH₃, CH₂CH₂CH₂C≡CCH₂CH₃, CH(CH₃)C≡CH, CH(CH₃)C≡CCH₃, CH(CH₃)C≡CCH₂Cl, CH(CH₃)C≡CCH₂CH₃, CH(CH₃)CH₂C≡CH, CH(CH₃)CH₂C≡CCH₃, CH(CH₃)CH₂C≡CCH₂Cl, CH(CH₃)CH₂C≡CCH₂CH₃, C(CH₃)₂C≡CH, C(CH₃)₂C≡CCH₃, C(CH₃)₂C≡CCH₂CH₃, CH(CF₃)C≡CH, CH(CF₃)C≡CCH₃, CH(CF₃)C≡CCH₂Cl, CH(CF₃)C≡CCH₂CH₃, CH(CF₃)CH₂C≡CH, CH(CF₃)CH₂C≡CCH₃, CH(CF₃)CH₂C≡CCH₂Cl, CH(CF₃)CH₂C≡CCH₂CH₃, which groups can be unsubstituted or substituted by one to three R^{a} groups.

Compounds of the formula I in which X is halogen (formula I.A), in particular chlorine, are an additionally preferred object of the invention.

Preference is given to compounds I in which the index m has the value 1, 2 or 3.

Preference is given to compounds I in which Lₘ represents fluorine, chlorine, methyl, C₁-haloalkyl, methoxy, amino, NHR or NR₂, in which R is methyl or acetyl.

In addition, particular preference is given to compounds I, wherein the phenyl group substituted by Lₘ is the group A 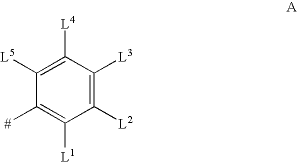
in which # is the point of linkage with the triazolopyrimidine skeleton and
L¹ represents fluorine, chlorine, CH₃ or CF₃;
L² and L⁴ represent, independently of one another, hydrogen or fluorine;
L³ represents hydrogen, fluorine, chlorine, CH₃, OCH₃, amino, NHR or NR₂; and
L⁵ represents hydrogen, fluorine or CH₃.

Special preference is given to compounds I, wherein Lₘ is one of the following substituent combinations: 2-fluoro-6-chloro, 2,6-difluoro, 2,6-dichloro, 2-fluoro-6-methyl, 2,4,6-trifluoro, 2,6-difluoro-4-methoxy, pentafluoro, 2-methyl-4-fluoro, 2-trifluoromethyl, 2-methoxy-6-fluoro, 2-chloro, 2-fluoro, 2,4-difluoro, 2-fluoro-4-chloro, 2-chloro-4-fluoro, 2,3-difluoro, 2,5-difluoro, 2,3,4-trifluoro, 2-methyl, 2,4-dimethyl, 2-methyl-4-chloro, 2-fluoro-4-methyl, 2,6-dimethyl, 2,4,6-trimethyl, 2,6-difluoro-4-methyl, 2-trifluoromethyl-4-fluoro, 2-trifluoromethyl-5-fluoro or 2-trifluoromethyl-5-chloro.

Special preference is given to compounds I in which X represents halogen or C₁-C₄-alkyl, such as chlorine or methyl, in particular chlorine.

Particular preference is given, in view of their use, to the compounds I compiled in the following tables. The groups mentioned in the tables for a substituent additionally represent, considered per se, independently of the combination in which they are mentioned, a particularly preferred form of the substituent in question.

Table 1

Compounds of the formula I in which X represents chlorine, Lₘ represents 2-fluoro-6-chloro and the combination of R¹ and R² for a compound corresponds in each case to a row of table A

Table 2

Compounds of the formula I in which X represents chlorine, Lₘ represents 2,6-difluoro and the combination of R¹ and R² for a compound corresponds in each case to a row of table A

Table 3

Compounds of the formula I in which X represents chlorine, Lₘ represents 2,6-dichloro and the combination of R¹ and R² for a compound corresponds in each case to a row of table A

Table 4

Compounds of the formula I in which X represents chlorine, Lₘ represents 2-fluoro-6-methyl and the combination of R¹ and R² for a compound corresponds in each case to a row of table A

Table 5

Compounds of the formula I in which X represents chlorine, Lₘ represents 2,4,6-trifluoro and the combination of R¹ and R² for a compound corresponds in each case to a row of table A

Table 6

Compounds of the formula I in which X represents chlorine, Lₘ represents 2,6-difluoro-4-methoxy and the combination of R¹ and R² for a compound corresponds in each case to a row of table A

Table 7

Compounds of the formula I in which X represents chlorine, Lₘ represents pentafluoro and the combination of R¹ and R² for a compound corresponds in each case to a row of table A

Table 8

Compounds of the formula I in which X represents chlorine, Lₘ represents 2-methyl-4-fluoro and the combination of R¹ and R² for a compound corresponds in each case to a row of table A

Table 9

Compounds of the formula I in which X represents chlorine, Lₘ represents 2-trifluoromethyl and the combination of R¹ and R² for a compound corresponds in each case to a row of table A

Table 10

Compounds of the formula I in which X represents chlorine, Lₘ represents 2-methoxy-6-fluoro and the combination of R¹ and R² for a compound corresponds in each case to a row of table A

Table 11

Compounds of the formula I in which X represents chlorine, Lₘ represents 2-chloro and the combination of R¹ and R² for a compound corresponds in each case to a row of table A

Table 12

Compounds of the formula I in which X represents chlorine, Lₘ represents 2-fluoro and the combination of R¹ and R² for a compound corresponds in each case to a row of table A

Table 13

Compounds of the formula I in which X represents chlorine, Lₘ represents 2,4-difluoro and the combination of R¹ and R² for a compound corresponds in each case to a row of table A

Table 14

Compounds of the formula I in which X represents chlorine, Lₘ represents 2-fluoro-4-chloro and the combination of R¹ and R² for a compound corresponds in each case to a row of table A

Table 15

Compounds of the formula I in which X represents chlorine, Lₘ represents 2-chloro-4-fluoro and the combination of R¹ and R² for a compound corresponds in each case to a row of table A

Table 16

Compounds of the formula I in which X represents chlorine, Lₘ represents 2,3-difluoro and the combination of R¹ and R² for a compound corresponds in each case to a row of table A

Table 17

Compounds of the formula I in which X represents chlorine, Lₘ represents 2,5-difluoro and the combination of R¹ and R² for a compound corresponds in each case to a row of table A

Table 18

Compounds of the formula I in which X represents chlorine, Lₘ represents 2,3,4-trifluoro and the combination of R¹ and R² for a compound corresponds in each case to a row of table A

Table 19

Compounds of the formula I in which X represents chlorine, Lₘ represents 2-methyl and the combination of R¹ and R² for a compound corresponds in each case to a row of table A

Table 20

Compounds of the formula I in which X represents chlorine, Lₘ represents 2,4-dimethyl and the combination of R¹ and R² for a compound corresponds in each case to a row of table A

Table 21

Compounds of the formula I in which X represents chlorine, Lₘ represents 2-methyl-4-chloro and the combination of R¹ and R² for a compound corresponds in each case to a row of table A

Table 22

Compounds of the formula I in which X represents chlorine, Lₘ represents 2-fluoro-4-methyl and the combination of R¹ and R² for a compound corresponds in each case to a row of table A

Table 23

Compounds of the formula I in which X represents chlorine, Lₘ represents 2,6-dimethyl and the combination of R¹ and R² for a compound corresponds in each case to a row of table A

Table 24

Compounds of the formula I in which X represents chlorine, Lₘ represents 2,4,6-trimethyl and the combination of R¹ and R² for a compound corresponds in each case to a row of table A

Table 25

Compounds of the formula I in which X represents chlorine, Lₘ represents 2,6-difluoro-4-methyl and the combination of R¹ and R² for a compound corresponds in each case to a row of table A

Table 26

Compounds of the formula I in which X represents chlorine, Lₘ represents 2-trifluoromethyl-4-fluoro and the combination of R¹ and R² for a compound corresponds in each case to a row of table A

Table 27

Compounds of the formula I in which X represents chlorine, Lₘ represents 2-trifluoromethyl-5-fluoro and the combination of R¹ and R² for a compound corresponds in each case to a row of table A

Table 28

Compounds of the formula I in which X represents chlorine, Lₘ represents 2-trifluoromethyl-5-chloro and the combination of R¹ and R² for a compound corresponds in each case to a row of table A

Table 29

Compounds of the formula I in which X represents chlorine, Lₘ represents 2,6-difluoro-5-cyano and the combination of R¹ and R² for a compound corresponds in each case to a row of table A

Table 30

Compounds of the formula I in which X represents chlorine, Lₘ represents 2,6-difluoro-4-methoxycarbonyl and the combination of R¹ and R² for a compound corresponds in each case to a row of table A

TABLE A

| No. | R¹ | R² |
|---|---|---|
| A-1 | H | CH₂C≡CH |
| A-2 | CH₃ | CH₂C≡CH |
| A-3 | CH₂CH₃ | CH₂C≡CH |
| A-4 | H | CH₂C≡CCH₃ |
| A-5 | CH₃ | CH₂C≡CCH₃ |
| A-6 | CH₂CH₃ | CH₂C≡CCH₃ |
| A-7 | H | CH₂C≡CCH₂Cl |
| A-8 | CH₃ | CH₂C≡CCH₂Cl |
| A-9 | CH₂CH₃ | CH₂C≡CCH₂Cl |
| A-10 | H | CH₂C≡CCH₂CH₃ |
| A-11 | CH₃ | CH₂C≡CCH₂CH₃ |
| A-12 | CH₂CH₃ | CH₂C≡CCH₂CH₃ |
| A-13 | H | CH₂CH₂C≡CH |
| A-14 | CH₃ | CH₂CH₂C≡CH |
| A-15 | CH₂CH₃ | CH₂CH₂C≡CH |
| A-16 | H | CH₂CH₂C≡CCH₃ |
| A-17 | CH₃ | CH₂CH₂C≡CCH₃ |
| A-18 | CH₂CH₃ | CH₂CH₂C≡CCH₃ |
| A-19 | H | CH₂CH₂C≡CCH₂CH₃ |
| A-20 | CH₃ | CH₂CH₂C≡CCH₂CH₃ |
| A-21 | CH₂CH₃ | CH₂CH₂C≡CCH₂CH₃ |
| A-22 | H | CH₂CH₂CH₂C≡CH |
| A-23 | CH₃ | CH₂CH₂CH₂C≡CH |
| A-24 | CH₂CH₃ | CH₂CH₂CH₂C≡CH |
| A-25 | H | CH₂CH₂CH₂C≡CCH₃ |
| A-26 | CH₃ | CH₂CH₂CH₂C≡CCH₃ |
| A-27 | CH₂CH₃ | CH₂CH₂CH₂C≡CCH₃ |
| A-28 | H | CH₂CH₂CH₂C≡CCH₂CH₃ |
| A-29 | CH₃ | CH₂CH₂CH₂C≡CCH₂CH₃ |
| A-30 | CH₂CH₃ | CH₂CH₂CH₂C≡CCH₂CH₃ |
| A-31 | H | CH(CH₃)C≡CH |
| A-32 | CH₃ | CH(CH₃)C≡CH |
| A-33 | CH₂CH₃ | CH(CH₃)C≡CH |
| A-34 | H | CH(CH₃)C≡CCH₃ |
| A-35 | CH₃ | CH(CH₃)C≡CCH₃ |
| A-36 | CH₂CH₃ | CH(CH₃)C≡CCH₃ |
| A-37 | H | CH(CH₃)C≡CCH₂Cl |
| A-38 | CH₃ | CH(CH₃)C≡CCH₂Cl |
| A-39 | CH₂CH₃ | CH(CH₃)C≡CCH₂Cl |
| A-40 | H | CH(CH₃)C≡CCH₂CH₃ |
| A-41 | CH₃ | CH(CH₃)C≡CCH₂CH₃ |
| A-42 | CH₂CH₃ | CH(CH₃)C≡CCH₂CH₃ |
| A-43 | H | CH(CH₃)CH₂C≡CH |
| A-44 | CH₃ | CH(CH₃)CH₂C≡CH |
| A-45 | CH₂CH₃ | CH(CH₃)CH₂C≡CH |
| A-46 | H | CH(CH₃)CH₂C≡CCH₃ |
| A-47 | CH₃ | CH(CH₃)CH₂C≡CCH₃ |
| A-48 | CH₂CH₃ | CH(CH₃)CH₂C≡CCH₃ |
| A-49 | H | CH(CH₃)CH₂C≡CCH₂Cl |
| A-50 | CH₃ | CH(CH₃)CH₂C≡CCH₂Cl |
| A-51 | CH₂CH₃ | CH(CH₃)CH₂C≡CCH₂Cl |
| A-52 | H | CH(CH₃)CH₂C≡CCH₂CH₃ |
| A-53 | CH₃ | CH(CH₃)CH₂C≡CCH₂CH₃ |
| A-54 | CH₂CH₃ | CH(CH₃)CH₂C≡CCH₂CH₃ |
| A-55 | H | C(CH₃)₂C≡CH |
| A-56 | CH₃ | C(CH₃)₂C≡CH |
| A-57 | CH₂CH₃ | C(CH₃)₂C≡CH |
| A-58 | H | C(CH₃)₂C≡CCH₃ |
| A-59 | CH₃ | C(CH₃)₂C≡CCH₃ |
| A-60 | CH₂CH₃ | C(CH₃)₂C≡CCH₃ |
| A-61 | H | C(CH₃)₂C≡CCH₂CH₃ |
| A-62 | CH₃ | C(CH₃)₂C≡CCH₂CH₃ |
| A-63 | CH₂CH₃ | C(CH₃)₂C≡CCH₂CH₃ |
| A-64 | H | CH(CF₃)C≡CH |
| A-65 | CH₃ | CH(CF₃)C≡CH |
| A-66 | CH₂CH₃ | CH(CF₃)C≡CH |
| A-67 | H | CH(CF₃)C≡CCH₃ |
| A-68 | CH₃ | CH(CF₃)C≡CCH₃ |
| A-69 | CH₂CH₃ | CH(CF₃)C≡CCH₃ |
| A-70 | H | CH(CF₃)C≡CCH₂Cl |
| A-71 | CH₃ | CH(CF₃)C≡CCH₂Cl |
| A-72 | CH₂CH₃ | CH(CF₃)C≡CCH₂Cl |
| A-73 | H | CH(CF₃)C≡CCH₂CH₃ |
| A-74 | CH₃ | CH(CF₃)C≡CCH₂CH₃ |
| A-75 | CH₂CH₃ | CH(CF₃)C≡CCH₂CH₃ |
| A-76 | H | CH(CF₃)CH₂C≡CH |
| A-77 | CH₃ | CH(CF₃)CH₂C≡CH |
| A-78 | CH₂CH₃ | CH(CF₃)CH₂C≡CH |
| A-79 | H | CH(CF₃)CH₂C≡CCH₃ |
| A-80 | CH₃ | CH(CF₃)CH₂C≡CCH₃ |
| A-81 | CH₂CH₃ | CH(CF₃)CH₂C≡CCH₃ |
| A-82 | H | CH(CF₃)CH₂C≡CCH₂Cl |
| A-83 | CH₃ | CH(CF₃)CH₂C≡CCH₂Cl |
| A-84 | CH₂CH₃ | CH(CF₃)CH₂C≡CCH₂Cl |
| A-85 | H | CH(CF₃)CH₂C≡CCH₂CH₃ |
| A-86 | CH₃ | CH(CF₃)CH₂C≡CCH₂CH₃ |
| A-87 | CH₂CH₃ | CH(CF₃)CH₂C≡CCH₂CH₃ |

The compounds I are suitable as fungicides. They are distinguished by an outstanding effectiveness against a broad spectrum of phytopathogenic fungi, especially from the classes of the Ascomycetes, Deuteromycetes, Oomycetes and Basidiomycetes. Some are systemically effective and they can be used in plant protection as foliar and soil fungicides.

They are particularly important in the control of a multitude of fungi on various cultivated plants, such as wheat, rye, barley, oats, rice, maize, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on fruit and vegetables,
Bipolaris and Drechslera species on cereals, rice and lawns,
Blumeria graminis (powdery mildew) on cereals,
Botrytis cinerea (gray mold) on strawberries, vegetables, ornamental plants and grapevines,
Erysiphe cichoracearum and Sphaerotheca fuliginea on cucurbits,
Fusarium and Verticillium species on various plants,
Mycosphaerella species on cereals, bananas and peanuts,
Phytophthora infestans on potatoes and tomatoes,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pyricularia oryzae on rice,
Rhizoctonia species on cotton, rice and lawns,
Septoria tritici and Stagonospora nodorum on wheat,
Uncinula necatoron grapevines,
Ustilago species on cereals and sugar cane, and
Venturia species (scab) on apples and pears.

The compounds I are also suitable for controlling harmful fungi, such as Paecilomyces variotii, in the protection of materials (e.g. wood, paper, paint dispersions, fibers or fabrics) and in the protection of stored products.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 0.001 to 0.1 g, preferably 0.01 to 0.05 g, per kilogram of seed are generally necessary.

When used in the protection of materials or stored products, the amount of active compound applied depends on the kind of application area and on the effect desired. Amounts customarily applied in the protection of materials are, for example, 0.001 g to 2 kg, preferably 0.005 g to 1 kg, of active compound per cubic meter of treated material.

The compounds I can be converted to the usual formulations, e.g. solutions, emulsions, suspensions, dusts, powders, pastes and granules. The application form depends on the respective use intended; it should always guarantee a fine and uniform distribution of the compound according to the invention.

The formulations are prepared in a known way, e.g. by extending the active compound with solvents and/or carriers, if desired using emulsifiers and dispersants, it being possible, when water is the diluent, also to use other organic solvents as auxiliary solvents. Suitable auxiliaries for this purpose are essentially: solvents, such as aromatics (e.g. xylene), chlorinated aromatics (e.g. chlorobenzenes), paraffins (e.g. petroleum fractions), alcohols (e.g. methanol, butanol), ketones (e.g. cyclohexanone), amines (e.g. ethanolamine, dimethylformamide) and water; carriers, such as ground natural minerals (e.g. kaolins, clays, talc, chalk) and ground synthetic ores (e.g. highly dispersed silicic acid, silicates); emulsifiers, such as nonionic and anionic emulsifiers (e.g. polyoxyethylene fatty alcohol ethers, alkylsulfonates and arylsulfonates) and dispersants, such as lignosulfite waste liquors and methylcellulose.

Suitable surfactants are alkali metal, alkaline earth metal and ammonium salts of lignosulfonic acid, naphthalenesulfonic acid, phenolsulfonic acid and dibutylnaphthalenesulfonic acid, alkylarylsulfonates, alkyl sulfates, alkylsulfonates, fatty alcohol sulfates and fatty acids, and alkali metal and alkaline earth metal salts thereof, salts of sulfated fatty alcohol glycol ethers, condensation products of sulfonated naphthalene and naphthalene derivatives with formaldehyde, condensation products of naphthalene or of naphthalenesulfonic acid with phenol and formaldehyde, polyoxyethylene octylphenol ethers, ethoxylated isooctylphenol, octylphenol and nonylphenol, alkylphenol polyglycol ethers, tributylphenyl polyglycol ethers, alkylaryl polyether alcohols, isotridecyl alcohol, fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Petroleum fractions having medium to high boiling points, such as kerosene or diesel fuel, furthermore coal tar oils, and oils of vegetable or animal origin, aliphatic, cyclic and aromatic hydrocarbons, e.g. benzene, toluene, xylene, paraffin, tetrahydronaphthalene, alkylated naphthalenes or derivatives thereof, methanol, ethanol, propanol, butanol, chloroform, carbon tetrachloride, cyclohexanol, cyclohexanone, chlorobenzene or isophorone, or highly polar solvents, e.g. dimethylformamide, dimethyl sulfoxide, N-methylpyrrolidone or water, are suitable for the preparation of directly sprayable solutions, emulsions, pastes or oil dispersions.

Powders, preparations for broadcasting and dusts can be prepared by mixing or mutually grinding the active substances with a solid carrier.

Granules, e.g. coated granules, impregnated granules and homogeneous granules, can be prepared by binding the active compounds to solid carriers. Solid carriers are, e.g., mineral earths, such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, e.g., ammonium sulfate, ammonium phosphate, ammonium nitrate or ureas, and plant products, such as cereal meal, tree bark meal, wood meal and nutshell meal, cellulose powders and other solid carriers.

The formulations generally comprise between 0.01 and 95% by weight, preferably between 0.1 and 90% by weight, of the active compound. The active compounds are employed therein in a purity of 90% to 100%, preferably 95% to 100% (according to the NMR spectrum).

Examples for formulations are:

I. 5 parts by weight of a compound according to the invention are intimately mixed with 95 parts by weight of finely divided kaolin. In this way, a dust comprising 5% by weight of the active compound is obtained.

II. 30 parts by weight of a compound according to the invention are intimately mixed with a mixture of 92 parts by weight of pulverulent silica gel and 8 parts by weight of liquid paraffin, which had been sprayed onto the surface of this silica gel. In this way, an active compound preparation with good adhesive properties (active compound content 23% by weight) is obtained.

III. 10 parts by weight of a compound according to the invention are dissolved in a mixture consisting of 90 parts by weight of xylene, 6 parts by weight of the addition product of 8 to 10 mol of ethylene oxide with 1 mol of the N-monoethanolamide of oleic acid, 2 parts by weight of the calcium salt of dodecylbenzenesulfonic acid and 2 parts by weight of the addition product of 40 mol of ethylene oxide with 1 mol of castor oil (active compound content 9% by weight).

IV. 20 parts by weight of a compound according to the invention are dissolved in a mixture consisting of 60 parts by weight of cyclohexanone, 30 parts by weight of isobutanol, 5 parts by weight of the addition product of 7 mol of ethylene oxide with 1 mol of isooctylphenol and 5 parts by weight of the addition product of 40 mol of ethylene oxide with 1 mol of castor oil (active compound content 16% by weight).

V. 80 parts by weight of a compound-according to the invention are intimately mixed with 3 parts by weight of the sodium salt of diisobutylnaphthalene-α-sulfonic acid, 10 parts by weight of the sodium salt of a lignosulfonic acid from a sulfite waste liquor and 7 parts by weight of pulverulent silica gel and are ground in a hammer mill (active compound content 80% by weight).

VI. 90 parts by weight of a compound according to the invention are mixed with 10 parts by weight of N-methyl-α-pyrrolidone and a solution is obtained which is suitable for use in the form of very small drops (active compound content 90% by weight).

VII. 20 parts by weight of a compound according to the invention are dissolved in a mixture consisting of 40 parts by weight of cyclohexanone, 30 parts by weight of isobutanol, 20 parts by weight of the addition product of 7 mol of ethylene oxide with 1 mol of isooctylphenol and 10 parts by weight of the addition product of 40 mol of ethylene oxide with 1 mol of castor oil. By running the solution into 100 000 parts by weight of water and finely dispersing it therein, an aqueous dispersion is obtained comprising 0.02% by weight of the active compound.

VIII. 20 parts by weight of a compound according to the invention are intimately mixed with 3 parts by weight of the sodium salt of diisobutylnaphthalene-α-sulfonic acid, 17 parts by weight of the sodium salt of a lignosulfonic acid from a sulfite waste liquor and 60 parts by weight of pulverulent silica gel and are ground in a hammer mill. A spray emulsion comprising 0.1% by weight of the active compound is obtained by fine dispersion of the mixture in 20 000 parts by weight of water.

The active compounds can be used as such, in the form of their formulations or of the application forms prepared therefrom, e.g. in the form of directly sprayable solutions, powders, suspensions or dispersions, emulsions, oil dispersions, pastes, dusts, preparations for broadcasting or granules, by spraying, atomizing, dusting, broadcasting or watering. The application forms depend entirely on the intended uses; they should

always guarantee the finest possible dispersion of the active compounds according to the invention.

Aqueous use forms can be prepared from emulsifiable concentrates, pastes or wettable powders (spray powders, oil dispersions) by addition of water. To prepare emulsions, pastes or oil dispersions, the substances can be homogenized in water, as such or dissolved in an oil or solvent, by means of wetting agents, tackifiers, dispersants or emulsifiers. However, concentrates comprising active substance, wetting agent, tackifier, dispersant or emulsifier and possibly solvent or oil can also be prepared, which concentrates are suitable for dilution with water.

The concentrations of active compound in the ready-for-use preparations can be varied within relatively wide ranges. In general, they are between 0.0001 and 10%, preferably between 0.01 and 1%.

The active compounds can also be used with great success in the ultra low volume (ULV) process, it being possible to apply formulations with more than 95% by weight of active compound or even the active compound without additives.

Oils of various types, herbicides, fungicides, other pesticides and bactericides can be added to the active compounds, if need be too not until immediately before use (tank mix). These agents can be added to the preparations according to the invention in a weight ratio of 1:10 to 10:1.

The preparations according to the invention can, in the application form as fungicides, also be present together with other active compounds, e.g. with herbicides, insecticides, growth regulators, fungicides or also with fertilizers. On mixing the compounds I or the preparations comprising them in the application form as fungicides with other fungicides, in many cases an expansion of the fungicidal spectrum of activity is obtained.

The following lists of fungicides, with which the compounds according to the invention can be used in conjunction, is intended to illustrate the possible combinations but not to limit them:
acylalanines, such as benalaxyl, metalaxyl, ofurace or oxadixyl,
amine derivatives, such as aldimorph, dodine, dodemorph, fenpropimorph, fenpropidin, guazatine, iminoctadine, spiroxamine or tridemorph,
anilinopyrimidines, such as pyrimethanil, mepanipyrim or cyprodinil,
antibiotics, such as cycloheximide, griseofulvin, kasugamycin, natamycin, polyoxin or streptomycin,
azoles, such as bitertanol, bromoconazole, cyproconazole, difenoconazole, diniconazole, epoxiconazole, fenbuconazole, fluquinconazole, flusilazole, flutriafol, hexaconazole, imazalil, metconazole, myclobutanil, penconazole, propiconazole, prochloraz, prothioconazole, tebuconazole, triadimefon, triadimenol, triflumizole or triticonazole,
dicarboximides, such as iprodione, myclozolin, procymidone or vinclozolin,
dithiocarbamates, such as ferbam, nabam, maneb, mancozeb, metam, metiram, propineb, polycarbamate, thiram, ziram or zineb,
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamide, thiophanate-methyl, tiadinil, tricyclazole or triforine,
copper fungicides, such as Bordeaux mixture, copper acetate, copper oxychloride or basic copper sulfate,
nitrophenyl derivatives, such as binapacryl, dinocap, dinobuton or nitrothal-isopropyl,
phenylpyrroles, such as fenpiclonil or fludioxonil,
sulfur,
other fungicides, such as acibenzolar-5-methyl, benthiavalicarb, carpropamid, chlorothalonil, cyflufenamid, cymoxanil, dazomet, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, hexachlorobenzene, metrafenone, pencycuron, propamocarb, phthalide, tolclofos-methyl, quintozene or zoxamide,
strobilurins, such as azoxystrobin, dimoxystrobin, fluoxastrobin, kresoxim-methyl, metominostrobin, orysastrobin, picoxystrobin, pyraclostrobin or trifloxystrobin,
sulfenic acid derivatives, such as captafol, captan, dichlofluanid, folpet or tolylfluanid,
cinnamamides and analogous compounds, such as dimethomorph, flumetover or flumorph.

### SYNTHESIS EXAMPLES

The procedures described in the following synthesis examples were used to prepare further compounds I by appropriate modification of the starting compounds. The compounds thus obtained are listed in the following tables, together with physical data.

### Example 1

### Preparation of 5-chloro-6-(2,4,6-trifluorophenyl)-7-propargylamino-[1,2,4]triazolo[1,5-a]pyrimidine [I-1]

A solution of 1.5 mmol of propargylamine and 1.5 mmol of triethylamine in 10 ml of dichloromethane was added, with stirring, to a solution of 1.5 mmol of 5,7-dichloro-6-(2,4,6-trifluorophenyl)[1,2,4]triazolo[1,5-a]pyrimidine [cf. WO 98/46607] in 20 ml of dichloromethane. The reaction mixture was stirred at 20-25° C. for approximately 16 hours and was then washed with dilute HCl solution. After separation of the phases, the organic phase was dried and freed from the solvent. After chromatographing the residue on silica gel, 0.42 g of the title compound was obtained, with a melting point of 141° C.

### Example 2

### Preparation of 5-cyano-6-(2,4,6-trifluorophenyl)-7-(N-methyl-N-propargylamino)[1,2,4]triazolo[1,5-a]pyrimidine

A mixture of 0.1 mol of the compound I-2 and 0.25 mol of tetraethylammonium cyanide in 750 ml of dimethylformamide (DMF) was stirred at 20-25° C. for approximately 16 hours. After addition of water and methyl tert-butyl ether (MTBE) and phase separation, the organic phase was washed with water, then dried and freed from solvents. After chromatographing the residue on silica gel, 4.72 g of the title compound were obtained, with a melting point of 147° C.

### Example 3

### Preparation of 5-methoxy-6-(2,4,6-trifluorophenyl)-7-(N-methyl-N-propargylamino)[1,2,4]triazolo[1,5-a]pyrimidine

A solution of 65 mmol of the compound I-2 in 400 ml of anhydrous methanol was treated with 71.5 mmol of sodium methoxide solution (30%) at 20-25° C. After stirring at this temperature for approximately 16 hours, the solvent was stripped off and the residue was taken up in dichloromethane. After washing with water, the organic phase was dried and then freed from solvent. After chromatographing the residue on silica gel, 3.94 g of the title compound were obtained, with a melting point of 119° C.

### Example 4

### Preparation of 5-methyl-6-(2,4,6-trifluorophenyl)-7-(N-methyl-N-propargylamino)[1,2,4]triazolo[1,5-a]pyrimidine

A mixture of 20 ml of diethyl malonate and 0.27 g (5.65 mmol) of sodium hydride (50% dispersion in mineral oil) in 50 ml of acetonitrile was stirred at 20-25° C. for approximately 2 hours. 4.7 mmol of the compound I-2 were added and then the mixture was stirred at 60° C. for approximately 20 hours. After addition of 50 ml of aqueous ammonium chloride solution, the mixture was acidified with dilute HCl solution and then extracted with MTBE. After drying, the combined organic phases were freed from the solvent. The crude product, after purifying by chromatographing on silica gel, was taken up in concentrated HCl and the mixture was then stirred at 80° C. for approximately 24 hours. After cooling, the reaction mixture was adjusted with aqueous NaOH solution to a pH of 5 and extracted with MTBE. After drying, the combined organic phases were freed from the solvent. After chromatographing the residue on silica gel, 0.62 g of the title compound was obtained.

¹H NMR (δ in ppm): 8.40 (s), 6.85 (m), 4.30 (d), 2.85 (s), 2.45 (s), 2.27 (s).

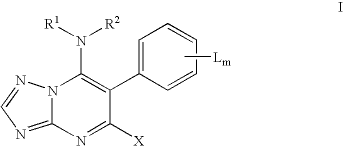

TABLE I

| | | | | | Phys. data |
|---|---|---|---|---|---|
| No. | R¹ | R² | X | Lₘ | (M.p. [° C.]) |
| I-1 | H | CH₂C≡CH | Cl | 2,4,6-F₃ | 141 |
| I-2 | CH₃ | CH₂C≡CH | Cl | 2,4,6-F₃ | 143 |
| I-3 | H | CH₂C≡CCH₂Cl | Cl | 2,4,6-F₃ | 173 |
| I-4 | H | C(CH₃)₂C≡CH | Cl | 2,4,6-F₃ | 227 |
| I-5 | H | CH₂C≡CH | Cl | 2-Cl-6-F | 190 |
| I-6 | H | C(CH₃)₂C≡CH | Cl | 2-Cl-6-F | 198 |
| I-7 | H | CH₂C≡CCH₃ | Cl | 2,4,6-F₃ | 210 |
| I-8 | H | CH(CH₃)C≡CH | Cl | 2,4,6-F₃ | 153 |
| I-9 | H | CH(CH₃)C≡CCH₃ | Cl | 2,4,6-F₃ | 66 |
| I-10 | H | CH₂C≡CCH₂CH₃ | Cl | 2,4,6-F₃ | 149 |
| I-11 | H | CH(CH₃)C≡CCH₂CH₃ | Cl | 2,4,6-F₃ | 89 |
| I-12 | H | CH₂CH₂C≡CH | Cl | 2,4,6-F₃ | 151 |
| I-13 | H | CH(CH₃)CH₂C≡CH | Cl | 2,4,6-F₃ | 140 |
| I-14 | H | CH₂CH₂C≡CCH₃ | Cl | 2,4,6-F₃ | 155 |
| I-15 | H | (CH₂)₃C≡CH | Cl | 2,4,6-F₃ | 152 |
| I-16 | H | CH(CH₃)C≡CH | Cl | 2-CH₃-4-F | 97 |
| I-17 | H | CH(CH₃)C≡CH | Cl | 2,4-F₂ | 106 |
| I-18 | H | CH(CH₃)C≡CH | Cl | 2-Cl-4-F | 108 |

Because of the hindered rotation of the phenyl group, two diastereoisomers may exist which may differ in their physical properties.

Examples for the action against harmful fungi

The fungicidal action of the compounds of the formula I can be demonstrated by the following tests:

The active compounds were prepared separately as a stock solution with 0.25% by weight of active compound in acetone or DMSO. 1% by weight of the emulsifier Uniperol® EL (wetting agent with an emulsifying and dispersing action based on ethoxylated alkylphenols) was added to this solution and appropriately diluted with water to the desired concentration.

### Use Example 1

### Activity Against Early Blight of Tomato Caused by Alternaria solani

Leaves of pot plants of the variety “Groβe Fleischtomate St. Pierre” were sprayed to runoff point with an aqueous suspension in the active compound concentration given below. On the following day, the leaves were infected with an aqueous suspension of spores of Alternaria solani in 2% Biomalz solution with a concentration of 0.17×10⁶ spores/ml. The plants were subsequently placed in a chamber saturated with water vapor at temperatures between 20 and 22° C. After 5 days, leaf infection in the untreated but infected control plants had so extensively developed that the infection could be visually determined in %.

In this test, the plants treated with 250 ppm of the active compounds Nos. I-3 and I-4 showed up to 3% infection, while the untreated plants were 80% infected.

### Use Example 2

### Curative Activity Against Wheat Leaf Rust Caused by Puccinia recondite

Leaves of wheat seedlings of the variety “Kanzler” grown in pots were dusted with spores of leaf rust (Puccinia recondite). The pots were afterwards placed in a chamber with high atmospheric humidity (90 to 95%) and 20 to 22° C. for 24 hours. During this time, the spores germinated and the germ tubes penetrated into the leaf tissue. The next day, the infected plants were sprayed to runoff point with an aqueous suspension in the active compound concentration given below. The suspension or emulsion was prepared from a stock solution with 10% of active compound in a mixture consisting of 89% of acetone and 1% of emulsifier. After the spray residue had dried on, the test plants were cultivated in a greenhouse at temperatures between 20 and 22° C. and a relative humidity of 65 to 70% for 7 days. The extent of the development of rust on the leaves was then determined.

In this test, the plants treated with 250 ppm of the active compounds Nos. I-3 and I-4 showed less than 3% infection, while the untreated plants were 80% infected.

1. 7-(Alkynylamino)triazolopyrimidines of the formula I 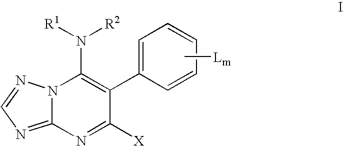
in which the substituents have the following meanings:
L is, independently of one another, halogen, C₁-C₆-alkyl, C₁-C₆-haloalkyl, C₁-C₆alkoxy, amino, NHR, NR₂, cyano, S(O)ₙA¹ or C(O)A²;
R is C₁-C₈-alkyl or C₁-C₈-alkylcarbonyl;
A¹ is hydrogen, hydroxyl, C₁-C₈-alkylamino or di(C₁-C₈-alkyl)amino;
n is 0, 1 or 2;
A² is C₂-C₈-alkenyl, C₁-C₈-alkoxy, C₁-C₆-haloalkoxy or one of the groups mentioned in A¹;
m is 1, 2, 3, 4 or 5, at least one L group being in the ortho position with respect to the bond with the triazolopyrimidine skeleton;
X is halogen, cyano, C₁-C₄-alkyl, C₁-C₄-haloalkyl or C₁-C₄-alkoxy;
R¹ is hydrogen or C₁-C₄-alkyl;
R² is C₃-C₁₀-alkynyl, which can be unsubstituted or partially or completely halogenated or can carry one to three R^{a} groups:
R^{a} is halogen, cyano, nitro, hydroxyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di(C₁-C₆-alkyl)amino, C₂-C₆-alkenyl, C₂-C₆-alkenyloxy, C₃-C₆-alkynyloxy or C₃-C₆-cycloalkyl,
these aliphatic or alicyclic groups for their part being able to be partially or completely halogenated or to carry one to three R^{b} groups;
R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl or dialkylaminothiocarbonyl, the alkyl groups in these radicals comprising 1 to 6 carbon atoms and the abovementioned alkenyl or alkynyl groups in these radicals comprising 2 to 8 carbon atoms.

2. Compounds of formula I.1 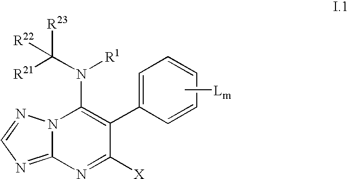
in which
R²¹ is methyl or halomethyl;
R²² is hydrogen, methyl or halomethyl;
R²³ is C₂-C₈-alkynyl, which can be unsubstituted or partially or completely halogenated and/or can carry one to three R^{a} groups;
and the other variables are defined as claimed in claim 1.

3. Compounds of formula I or I.1 as claimed in claim 1 or 2, wherein X represents chlorine or methyl, in particular chlorine.

4. Compounds of formula I or I.1 as claimed in claim 1, wherein the phenyl group substituted by Lₘ is the group A 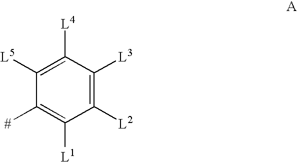
in which # is the point of linkage with the triazolopyrimidine skeleton and
L¹ represents fluorine, chlorine, CH₃ or CF₃;
L² and L⁴ represent, independently of one another, hydrogen or fluorine;
L³ represents hydrogen, fluorine, chlorine, CH₃, OCH₃, amino, NHR or NR₂; and
L⁵ represents hydrogen, fluorine or CH₃.

5. Compounds of formula I as claimed in claim 1, wherein the phenyl group substituted by Lₘ is one of the following substituent combinations: 2-fluoro-6-chloro, 2,6-difluoro, 2,6-dichloro, 2-fluoro-6-methyl, 2,4,6-trifluoro, 2,6-difluoro-4-methoxy, pentafluoro, 2-methyl-4-fluoro, 2-trifluoromethyl, 2-methoxy-6-fluoro, 2-chloro, 2-fluoro, 2,4-difluoro, 2-fluoro-4-chloro, 2-chloro-4-fluoro, 2,3-difluoro, 2,5-difluoro, 2,3,4-trifluoro, 2-methyl, 2,4-dimethyl, 2-methyl-4-chloro, 2-fluoro-4-methyl, 2,6-dimethyl, 2,4,6-trimethyl, 2,6-difluoro-4-methyl, 2-trifluoromethyl-4-fluoro, 2-trifluoromethyl-5-fluoro or 2-trifluoromethyl-5-chloro.

6. A process for the preparation of the compound of the formula I as claimed in claim 1 by reaction of dihalotriazolopyrimidines of the formula II 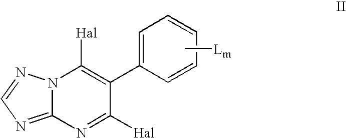
in which the variables have the meanings given for formula I and Hal is a halogen atom, in particular chlorine, with amines of the formula III

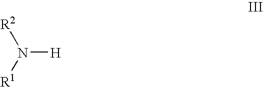

7. A preparation suitable for the control of harmful fungi, comprising a solid or liquid carrier and a compound of the formula I as claimed in claim 1.

8. A process for the control of harmful phytopathogenic fungi, which comprises treating the fungi or the materials, plants, ground or seeds to be protected from fungal attack with an effective amount of a compound of the formula I as claimed in claim 1.
